(19) United States
(12) Patent Application Publication
(10) Pub. No.: US 2010/0241093 A1
(43) Pub. Date: Sep. 23, 2010

(54) OSTOMY POUCHING SYSTEM

(76) Inventor: David Leonard HOOPER, Langley (CA)

Correspondence Address: VERMETTE & CO., SUITE 320 - 1177 WEST HASTINGS STREET, VANCOUVER, BC, V6E2K3, CA

(21) Appl. No.: 12/406,758

(22) Filed: Mar. 18, 2009

(51) Int. Cl. A61F 5/445

(52) U.S. Cl. 604/339

(57) ABSTRACT

An ostomy pouching system having an ostomy wafer with a sealing ring around an opening therein and an ostomy bag having an opening with a sealing ring around the ostomy bag opening, the ostomy bag sealing ring sealingly connectable to the sealing ring of the ostomy wafer. An insert is insertable into the ostomy bag through the ostomy bag opening. The insert has an opening with a flange surrounding the opening and extending flat over the sealing ring of the ostomy bag so that upon the sealing ring of the ostomy bag engaging the sealing ring on the wafer, the flange is sealed between the ostomy bag sealing ring and the wafer sealing ring.

### FIELD

The present invention relates to an ostomy pouching system having a wafer which attaches to a user and having an opening over the stoma or opening of the colon which attaches to the abdominal wall and an ostomy bag which sealingly engages the wafer. An insert which fits through the ostomy bag and is sealed between the latter bag and the wafer, fills with waste and is then discarded without having to dispose of the much costlier ostomy bag.

### BACKGROUND

An ostomy pouching system is a medical prosthetic that provides a means for the collection of waste from a diverted biological system (colon, ileum, urinary). Pouching systems are most commonly associated with colostomies, ileostomies and urostomies. For example, in a colostomy (also called an ostomy), a surgically created opening of the large intestine or colon which results in a stoma, is created when a portion of the colon or the rectum is removed and the remaining colon is brought to the abdominal wall where an opening is formed in the wall to allow the end of the colon to discharge waste through the opening. An ostomy pouching system is used to control the waste after it is discharged.

Pouching systems usually consist of a mounting plate, commonly called a wafer, and a collection bag that is mechanically attached in an airtight seal. The pouching systems collect waste that is output from a stoma it allows the stoma to drain into a sealed collection bag, while protecting the surrounding skin from contaminaton. Pouching systems come in one- and two-piece designs; two-piece designs allow the changing and use of multiple bags while retaining the wafer for several days. The cost of each bag is approximately $5.00 so that changing the bag after each discharge is expensive. It would be desirable to have an inexpensive insert that could be used and discarded rather than discarding the whole bag.

### SUMMARY OF THE INVENTION

According to the invention there is provided an ostomy pouching system having an ostomy wafer with a sealing ring around an opening therein and an ostomy bag having an opening with a sealing ring around the ostomy bag opening, the ostomy bag sealing ring being sealingly connectable to the sealing ring of the ostomy wafer. An insert is insertable into the ostomy bag through the ostomy bag opening. The insert has an opening with a flange surrounding the opening and extending flat over the sealing ring of the ostomy bag so that upon the sealing ring of the ostomy bag engaging the sealing ring on the wafer, the flange is sealed between the ostomy bag sealing ring and the wafer sealing ring.

A diameter of the insert when filled is less than or equal to the diameter of the ostomy bag opening.

Advantageously, the ostomy bag is water dispersible.

The ostomy bag may be biodegradable.

### BRIEF DESCRIPTION OF THE DRAWINGS

Further features and advantages will be apparent from the following detailed description, given by way of example, of a preferred embodiment taken in conjunction with the accompanying drawings, wherein:

FIG. 1 is an ostomy bag with an opening, a flange surrounding the opening and a sealing ring around the flange;

FIG. 2 is a side view of the bag of FIG. 1;

FIG. 3 shows a bag and wafer attached to a user;

FIG. 4 is a wafer having an opening surrounded by a sealing ring;

FIG. 5 is a wafer attached to a user;

FIG. 6 is an exploded view of a user with a wafer attached and an insert with the ostomy bag;

FIG. 7 shows the ostomy bag and insert inside the pouch attached to the wafer; and

FIG. 8 is an insert.

### DETAILED DESCRIPTION WITH REFERENCE TO THE DRAWINGS

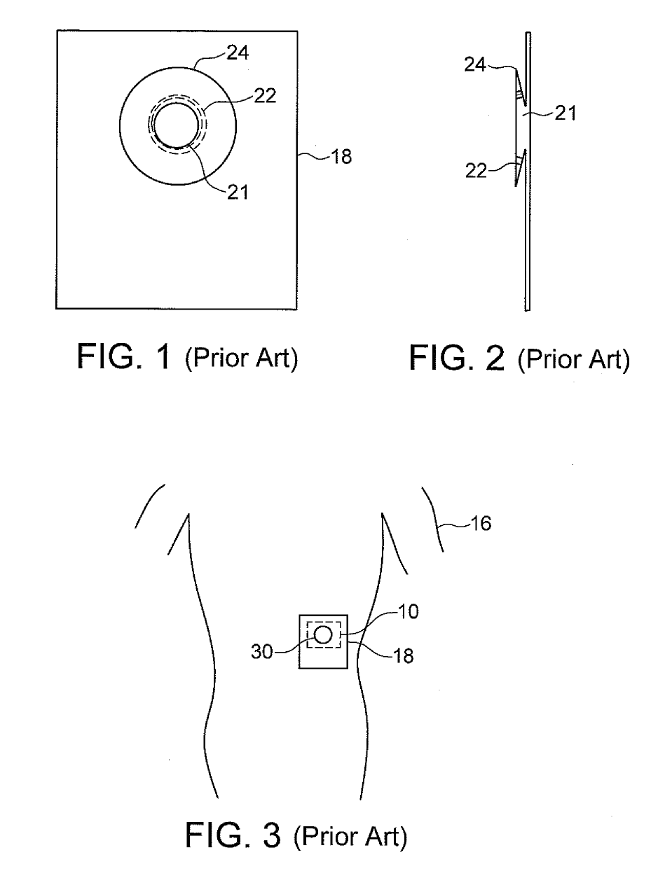

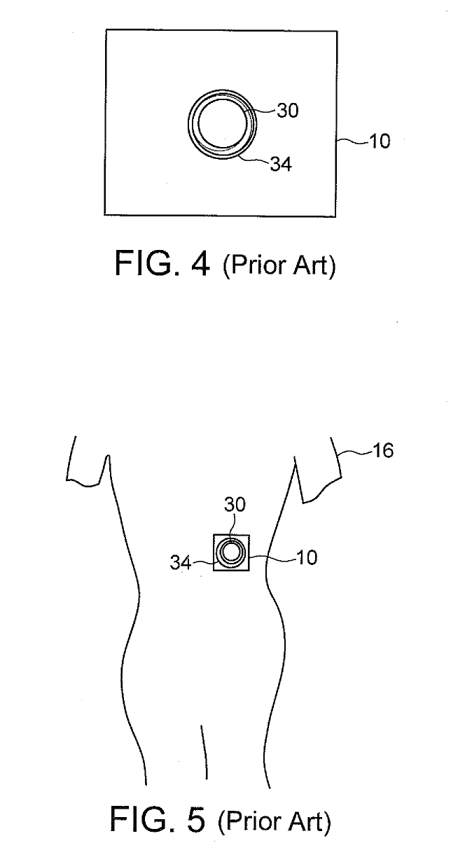

Referring to FIG. 1 there is shown a conventional ostomy bag 18 having a hole 21 surrounded by a flange 24 with a sealing ring 22. FIG. 2 is an edge view of the ostomy bag 18. FIG. 3 shows a user 16 with an ostomy 18 attached to a wafer 10 which, in turn, is attached to a user 16. Typically, the body facing side of the wafer has an adhesive which adheres to the skin of a user, but is also removable much like surgical tape. The wafer has a hole 30 which is positioned over the opening in the abdominal wall around which the open end of the colon is attached. A sealing ring on the wafer engages and seals with a mating sealing ring on the flange of the ostomy bag 18. FIG. 4 shows a wafer 10 which has a releasable adhesive on its body facing surface and an opening 30 surrounded by a releasable seal. FIG. 5 shows a user 16 with the wafer 10 attached.

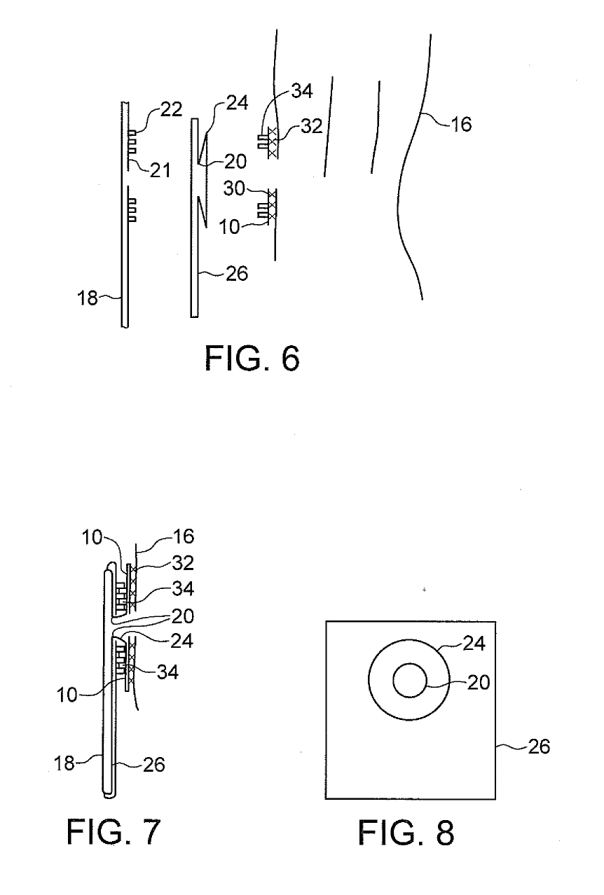

Referring to FIG. 8 an insert is shown having an opening 20 and a flange 24 heat sealed to the bag around the opening 20. Typically the insert is of plastic but may be made with a more complex structure if it is to be water soluble. If the opening 20 is 2½ inches, then the width of the bag when flat should be no more than about 4 inches in order to be pulled through the opening 20 when nearly full. Referring to FIG. 6 the wafer 10 is attached to a user 16 with a releasable adhesive 32 and its sealing ring 34 facing outwardly. Insert 26 is then passed through opening 21 of ostomy bag 18 with only flange 24 left outside. Flange 24 is laid flat over sealing ring 22 and then the latter is pressed into engagement with sealing ring 34 sandwiching the insert flange 24 in between as shown in FIG. 7.

When waste is discharged from the stoma it enters the insert rather than the ostomy bag 18. Any odor is prevented from being released by the tight seal of the sealing rings 21 and 34 with the insert flange 24 flat against the sealing rings 21 and 34. When the insert 26 is full, the bag 18 is pulled away from wafer 10 and the insert 26 is discarded. Since ordinary plastic is insoluble in water, the bags cannot be disposed of in the toilet. It is possible to manufacture an insert with a more complicated structure that is dispersible in water. For example, one could make a bag from water soluble material by making that material water resistant on the inside and water soluble on the outside. While in contact with the waste material there would be only a slow breakdown of the material. However, once the material is contacted by water, a more rapid dispersion begins. A paper core with a coating such as polyvinyl dylene chloride (PVDC) can be used on the inside of the core for its low transmissibility for water and polyvinyl alcohol (PVOH) on the outside of the core for its solubility in water.

Accordingly while this invention has been described with reference to illustrative embodiments, this description is not intended to be construed in a limiting sense. Various modifications of the illustrative embodiment will be apparent to those skilled in the art upon reference to this description. It is therefore contemplated that appended claims will cover any such modifications or embodiments as fall within the scope of the invention.

I claim:

1. An ostomy pouching system having an ostomy wafer with a sealing ring around an opening therein and an ostomy bag having an opening with a sealing ring around said ostomy bag opening, said ostomy bag sealing ring sealingly connectable to said sealing ring of said ostomy wafer, comprising: an insert insertable into said ostomy bag through said ostomy bag opening, said insert having an opening with a flange surrounding said opening and extending flat over said sealing ring of said ostomy bag so that upon the sealing ring of said ostomy bag engaging the sealing ring on said wafer, said flange is sealed between said ostomy bag sealing ring and said wafer sealing ring.

2. The system of claim 1, wherein a diameter of said insert when filled is less than or equal to the diameter of said ostomy bag opening.

3. The system of claim 1, wherein said ostomy bag is water dispersible.

4. The system of claim 1, wherein said ostomy bag is biodegradable.

5. An ostomy pouching system, comprising:
(a) a wafer having a releasable adhesive on a back side and an opening with a sealing ring enclosing said opening on a front side thereof;
(b) an ostomy bag having an opening of substantially the same dimensions as said wafer and a sealing ring around said ostomy bag opening which mates and engages the sealing ring on said wafer;
(c) an insert insertable into said ostomy bag through said ostomy bag opening, said insert having an opening with a flange surrounding said opening and extending flat over said sealing ring of said ostomy bag so that upon the sealing ring of said ostomy bag engaging the sealing ring on said wafer, said flange is sealed between said ostomy bag sealing ring and said wafer sealing ring.

6. The system of claim 5, wherein a diameter of said insert when filled is less than or equal to the diameter of said ostomy bag opening.

7. The system of claim 5, wherein said ostomy bag is water dispersible.

8. The system of claim 5, wherein said ostomy bag is biodegradable.

9. An ostomy pouching system for use with a wafer having a releasable adhesive on a back side and an opening with a sealing ring enclosing said opening on a front side thereof, comprising:
(a) an ostomy bag having an opening of substantially the same dimensions as said wafer and a sealing ring around said ostomy bag opening which mates and engages the sealing ring on said wafer;
(b) an insert insertable into said ostomy bag through said ostomy bag opening, said insert having an opening with a flange surrounding said opening and extending flat over said sealing ring of said ostomy bag so that upon the sealing ring of said ostomy bag engaging the sealing ring on said wafer, said flange is sealed between said ostomy bag sealing ring and said wafer sealing ring.
